(19) United States
(12) Patent Application Publication
(10) Pub. No.: US 2021/0402212 A1
(43) Pub. Date: Dec. 30, 2021

(54) ANTI-MICROBIAL PHOTOBIOMODULATION SUBSTRATE

(71) Applicant: DMC Limited L.C., Sheridan, WY (US)

(72) Inventors: Charles Joseph Schupp, Minnetrista, MN (US); John Mario Sartori, Minnetrista, MN (US); Will Schupp, Sheridan, WY (US)

(21) Appl. No.: 17/358,757

(22) Filed: Jun. 25, 2021

Related U.S. Application Data
(60) Provisional application No. 63/044,609, filed on Jun. 26, 2020.

(51) Int. Cl. A61N 5/06; A61N 5/067

(52) U.S. Cl. CPC A61N 5/0624; A61N 5/067; A61N 2005/0663; A61N 2005/0648; A61N 2005/0651; A61N 2005/0632

(57) ABSTRACT

A method includes applying a substrate containing light emitting devices to an individual's body part and controlling the light emitting devices to deliver light in an antimicrobial amount to the body part, wherein the light has a wavelength in the range of 400 nm to 1300 nm and an irradiance of from 2 mW-100 mW/cm2.

### CROSS-REFERENCE TO RELATED APPLICATIONS

This application claims the benefit of the filing date of U.S. application No. 63/044,609, filed on Jun. 26, 2020, the disclosure of which is incorporated by reference herein.

### BACKGROUND

Light has been used in various methods of medical treatment, such as to enhance healing of bums and other medical conditions. Light therapy has included exposure to daylight or to specific wavelengths of light using polychromatic polarized light, lasers, light-emitting diodes, fluorescent lamps, dichroic lamps or very bright, full-spectrum light. The light is administered for a prescribed amount of time and, in some cases, at a specific time of day.

One common use of the term is associated with the treatment of skin disorders, chiefly psoriasis, acne vulgaris, eczema and neonatal jaundice.

Light therapy which strikes the retina of the eyes is used to treat diabetic retinopathy and also circadian rhythm disorders such as delayed sleep phase disorder.

Photobiomodulation (PBM) has been proposed, researched and utilized for treatment of various disease states with proven benefits enhancing healing in a variety of organ systems including hair regrowth, myriad of skin conditions, wound healing and improved perfusion in experimental models of ischemia.

More recently, enhancement of normal tissue bioenergetic and cellular function has been proposed and researched with proven benefits for both performance and recovery utilized before and/or after in a variety of laboratory exercise tests.

There are several devices on the market utilizing both LED and low-level laser (LLL) therapy as either a hand-held or total body enclosure treatment system, and/or for healing/treatment of diseases or disease states as well as athletic performance.

### SUMMARY

Light producing mechanisms are embedded in, or associated with, a device (substrate), e.g., a patch, that in one embodiment is in contact with, e.g., applied or adhered to, skin of a mammal, and in one embodiment, directed at physiological sites including lung tissue and/or specific muscle groups to stimulate on a cellular, subcellular and optionally systemic level the enhancement of nitric oxide, thereby providing an antimicrobial effect. The mammal may be infected with a microbe, or may be at risk of being infected, e.g., after exposure to a microbial source including but not limited to an infected mammal or a mammal showing signs of infection such as a fever and/or other characteristics of infection with a microbe, e.g., dry cough, loss of taste or scent, or difficulty breathing, or any combination thereof. A mammal having one or more of the substrates may be monitored for signs of infection, e.g., before and/or after delivering a dose of light to, for example, monitored and based on that monitoring, initiating light delivery or monitoring the effectiveness of the light delivery. For example, near infrared spectrometry (NIRS) may be employed to detect nitrosylated hemoglobin, oxygenated hemoglobin, deoxygenated hemoglobin, pulse rate, pulse variability, the activity and density of porphyrins, and/or the redox state of cytochrome c oxidase.

Also provided is the use of the device to treat physiological samples ex vivo, including physiological fluid samples such as blood. In one embodiment, the device is used in conjunction with extracorporeal membrane oxygenation (ECMO).

Light therapy can also be used for prevention of migraines, to treat seasonal affective disorder, and for treatment of non-seasonal psychiatric disorders.

### BRIEF DESCRIPTION OF THE DRAWINGS

FIG. 1 is a block diagram of a system using photobiomodulation for performance enhancement according to an example embodiment.

FIG. 2 is a block diagram illustrating a substrate having electronics and light actuators according to an example.

FIG. 3 illustrates a view of a substrate within the scope of the disclosure incorporating a system for enhancing NO levels in a mammal such as a human according to an example embodiment.

FIG. 4 is block cross sectional representation of multiple different arrangements of light sources according to an example embodiment.

FIG. 5 is a flowchart illustrating a computer implemented method 500 use and operation of a substrate according to an example embodiment.

FIG. 6 is a block diagram illustrating circuitry for controlling lights to allow for an antimicrobial effect in methods according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

Various devices and methods are described herein that utilize photobiomodulation (PBM) in substrates (external devices) that emit energy in an amount the inhibits the in vivo replication of utilizing arrays of light emitting diodes (LEDs), low level laser light (LLL), or multitudes of both functioning at specific bandwidths with pulsed or continuous electromagnetic energy in the visible and near infrared portion of the light. Light can act on different mechanisms within cellular tissue to stimulate or suppress biological activity, e.g., suppress the replication of microbes, in a process commonly referred to as photobiomodulation (PBM). PBM involves the use of visible to near infrared light (NIR) (400-1300 nm) produced by a laser or a non-coherent light source applied to the surface of the body and has been shown to produce beneficial effects, and as described herein, beneficial effects on mammals infected with a microbe, e.g., a virus such as SARS-CoV2.

PBM utilizes light with a suitable intensity, energy, and wavelengths, without significantly causing damage to the cells. Some photo-acceptors, such as water or hemoglobin, are ubiquitous and absorb light to such a degree that little or no penetration of light energy into a tissue occurs. For example, water absorbs light most willingly above approximately 1300 nanometers. Thus, energy in this range has little ability to penetrate tissue due to the water content. However, water is more transparent in wavelengths between 400 and 1300 nanometers. Another example is hemoglobin, which absorbs heavily in the region between 300 and 670 nanometers but is reasonably transparent above approximately 670 nanometers.

Based on these assumptions, one can define a “spectral window” into the body. Within the window, there are certain wavelengths that are more or less likely to penetrate and target a specific chromophore and resultant target effect. The mechanism of PBM at the cellular level has been ascribed to the activation of mitochondrial respiratory chain components resulting in stabilization of metabolic function. A growing body of evidence suggests that the mitochondrial enzyme cytochrome C oxidase (CCO) is a key photo-acceptor of light in the far red to near infrared spectral range. These specific chromophores or photo-acceptors in the target tissue, most notably CCO, and nitric oxide (NO), may have unique interdependent roles in controlling cellular energy production and maintenance of cellular redox status.

Under certain conditions such as hypoxia or cellular fatigue, NO is produced, which has an inhibitory effect on the electron transport chain (ETC) by binding to and deactivating CCO which is the final electron acceptor of the ETC. Light therapy has been shown to dissociate bound NO from CCO and other cellular sites such as myoglobin, thus allowing continued ATP production via restoration of the mitochondrial membrane potential either directly by CCO activity or by activation of light or heat-gated ion channels. In addition to enhanced respiration via the ETC, relatively small concomitant increase in reactive oxygen species (ROS) production has been shown in vitro and vivo with light application.

ROS in healthy cells can provide enhanced cellular signaling and stimulation of nuclear transcription factors including nuclear transcription factor kappa beta, which can result in transcription of various proteins which can in turn provide cellular protection against oxidative stress caused by increased. ROS. In exercising tissue, this upregulation of transcription factors may explain the chronic and long-term beneficial effects of PBM stemming from improved adaptation.

In various embodiments of the present inventive subject matter, light producing mechanisms are embedded in, or associated with, substrates that are in proximity to a physiological area, e.g., the lung or specific muscle groups/skin sites, of a mammal such as a human, to stimulate on a cellular, subcellular and optionally a systemic level an anti-microbial effect, e.g., as a result of an increase in NO or iNOS that is stimulated by the application of light. Though specific physiological sites may be targeted and are benefited, localized PBM therapy has systemic effects as well.

In one embodiment, combinations or single LED's/LLL's are embedded into the substrate.

These light sources may have predetermined but adjustable parameters including but not limited to wavelength, pulse width, duration, dosage, and frequency. In at least sonic embodiments, each preselected wavelength of the light is selected to be at or near a transmission peak (or at or near an absorption minimum) for the intervening tissue. In at least some embodiments, one wavelength corresponds to a peak in the transmission spectrum of tissue at or near 850 nanometers (NIR). In at least some embodiments, one wavelength corresponds to a peak in the transmission spectrum of tissue at or near 650 nanometers (red visible).

Other specific embodiments use different means of light delivery, including OLEDs, microLEDs, or low-level lasers. Other specific embodiments also employ different means of embedding the light delivery mechanisms (e.g., LEDs) into the substrates including glue, adhesive tape, snaps, stitching, heat activated seam tape, Velcro brand hook and loop fasteners, weaving the light delivery mechanisms into the fabric itself, or printing the light delivery mechanisms onto a flexible substrate embedded in or mounted on the fabric. A heat regulating phase change material can be added between the lights and the skin to regulate the temperature of the light delivery system itself, as well as to regulate the temperature of the subject using the lights by conduction of heat, combined with the vasodilation caused by the lights themselves. This regulation of temperature can have a separate therapeutic effect and has been shown to improve athletic performance and fatigue.

These parameters may be controlled via external and/or attached device by way of internal microprocessors and controllers. This external device could be but is not limited to smartphones, sports devices (e.g., sports watches or bike computers), or proprietary devices.

Parameters may be controlled manually or automatically, but parameter values may be limited to be set within predefined safe ranges. In at least some embodiments, the temporal profile (e.g., peak irradiance, temporal pulse width, and duty cycle) is selected to utilize the kinetics of the biological processes while maintaining the irradiated portion of the tissue at or below a predetermined temperature.

In at least some embodiments, the pulse energy density, or energy density per pulse, can be calculated as the time-averaged power density divided by pulse repetition rate, or frequency. For example, the smallest pulse energy density will happen at the smallest average power density and fastest pulse repetition rate, where the pulse repetition rate is duty cycle divided by the temporal pulse width, and the largest pulse energy density will happen at the largest average power density and slowest pulse repetition rate. Conditional language, for example, among others, “can,” “could,” “might,” or “may,” unless specifically stated otherwise, or otherwise understood within the context as used, is generally intended to convey that certain embodiments include, while other embodiments do not include, certain features, elements or steps.

In one embodiment, the disclosure provides for a method comprising: subjecting a mammalian physiological fluid sample ex vivo to an antimicrobial amount of light emitted from one or more light emitting devices, wherein the light has a wavelength in the range of 400 nm to 1300 nm. In one embodiment, the method employs a total dose of irradiance that is no more than 100 mW/cm2. In one embodiment, the mammal is a human. In one embodiment, one or more substrates have one or more light emitting devices.

While the present disclosure has been discussed in the context of certain embodiments and examples, it should be appreciated that the present disclosure extends beyond the specifically disclosed embodiments to other alternative embodiments or uses of the present disclosures and obvious modifications and equivalents thereof.

Components can be added, removed, or rearranged. Additionally, the skilled artisan will recognize that any of the above-described methods can be carried out using any appropriate apparatus. Further, the disclosure herein of any particular feature, aspect, method, property, characteristic, quality, attribute, element, or the like in connection with various embodiments can be used in all other embodiments set forth herein. Additionally, processing steps may be added, removed, or reordered. A wide variety of designs and approaches are possible.

For purposes of this disclosure, certain aspects, advantages, and novel features of the present disclosure are described herein. It is to be understood that not necessarily all such advantages may be achieved in accordance with any particular embodiment of the present disclosure. Thus, for example, those skilled in the art will recognize that the present disclosure may be embodied or carried out in a manner that achieves one advantage or group of advantages as taught herein without necessarily achieving other advantages as may be taught or suggested herein.

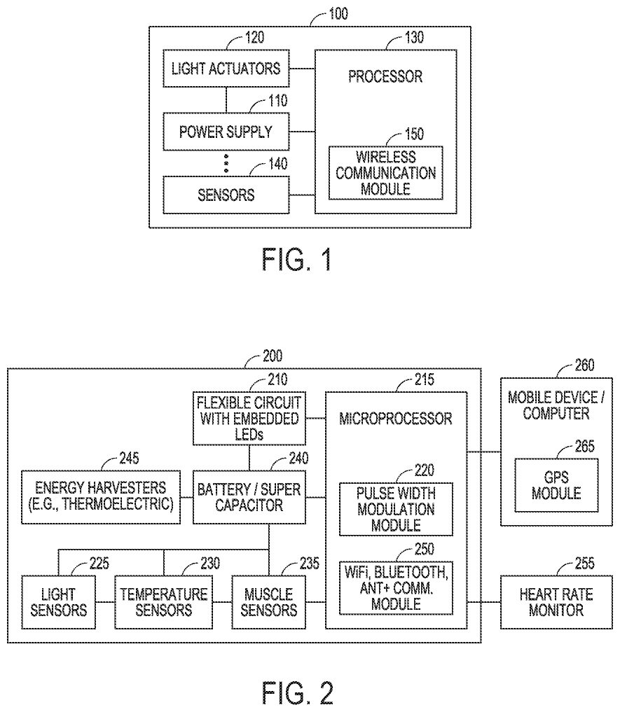

FIG. 1 is a block diagram of a system 100 using photobiomodulation for performance enhancement according to an example embodiment. In one embodiment, the system 100 is integrated or otherwise supported or held in place in a substrate. System 100 includes a power supply 110, one or more light sources, referred to as light actuators 120 and a controller 130 coupled to the power supply 110 and light actuators 120. The controller 130 controls the light actuators 120 to deliver light to targeted areas on the body as a function of the substrate position on the body.

The light actuators 120 can include an array of one or more LEDs, microLEDs, oLEDs, and LLL technology. The system 100 used an embedded controller 130, which may include a processor, such as a microprocessor that receives sensor readings via sensors 140, performs computations, controls actuators, and performs communication via wired or wireless communications module 150. Some functions of the controller 130 include control of pulse width modulation, dosimetry, spectral switching, communication with external devices such as phones, tablets, computers, smart watches, etc., data logging, interface with software. Optional external or internal sensors 140 include, but are not limited to light sensor, EKG, power meter, lactate monitoring, muscle oxygen sensors, stretch sensor/strain gauge. Further sensors associated with localized PBM to enhance NO and/or INOS production include one or more of near-infrared spectroscopy sensors to measure nitric oxide, blood oxygenation sensors, muscle oxygenation sensors, heart rate/rate variability sensors, temperature sensors to measure body temperature, and audio or accelerometer sensors to measure auscultation of the lungs for findings of antimicrobial effects as well as detecting respiratory rate.

A communication module is used to communicate with external devices or other components. Communication protocols include but are not limited to Bluetooth, ant+, and/or Wi-Fi. Electrical components are ordered by the power supply 110, which may be a rechargeable battery, super-capacitor, and/or energy harvester. In some embodiments, the system 100 in the form of a substrate may have a rectangular shape that is large enough to provide light to selected parts of the body. The light actuators 120 may include an array of lights that may be controlled by the controller 130 to illuminate a selected body part by providing electricity to light actuators positioned about the selected part such that the light is directed to the selected body part. In further embodiments, arrays may be shaped to illuminate a selected body part with all light actuators providing illumination. In one embodiment, the system may include near infrared light sensors. This may be employed to detect nitrosylated hemoglobin, oxygenated hemoglobin, deoxygenated hemoglobin, pulse rate, pulse variability, the activity and density of porphyrins, and/or the redox state of cytochrome c oxidase.

FIG. 2 is a block diagram illustrating a substrate item 200 having electronics and light actuators according to an example embodiment.

In one embodiment, substrate 200 includes multiple components embedded into a substrate such as one formed of fabric. The embedded components may include LEDs of various wavelengths are mounted on a flexible circuit board or light delivery mechanism 210 that is attached to the substrate 200 via stitching, snaps, glue, or Velcro brand hook and loop fasteners. The LEDs are controlled by an embedded microcontroller 215 that uses a pulse width modulation module 220 to control a power level and dose delivered by the LEDs. The microcontroller 215 also interfaces with various sensors 225, 230, 235 to determine appropriate dosing parameters—for example, sensors 235 measure muscle contractions that help to determine optimal time between doses, temperature sensors 230 monitor temperature and prevent overheating, and light sensors 225, e.g., near infrared light spectroscopy (NIRS), provide light measurement that may be used to adjust the applied dosage for various skin types and body compositions.

The electrical components embedded into the substrate are powered by an embedded battery or super-capacitor 240. In some embodiments, the battery/supercapacitor 240 is fed by energy harvesters 245 such as thermoelectric energy harvesters that harvest energy from surroundings and from the body itself to charge the battery/super-capacitor 240. The microcontroller 215 also contains a wireless communication module 250 that is capable of communication with external devices via Wi-Fi, Bluetooth, and ANT+ communication protocols. Examples of such compatible devices are heart rate monitors 255—used to monitor a user's heart rate to adjust applied dosage—and mobile phones 260—used to communicate user inputs to the microcontroller 215 to control program parameters such as user characteristics, selection of manual or automatic dosing, desired dose levels and times, etc.

Communication with phone 260 that may have an embedded or external GPS modules 265 can also he used to gather information about the mammal, which can be used to determine when and at what intensity to apply doses. Communication with mobile devices or computers can also be used to capture statistics, including time, intensity, and duration of applied doses. Other specific embodiments use different means of light delivery mechanism 210, including OLEDs, microLEDs, or low-level lasers. Other specific embodiments also employ different means of embedding the light delivery mechanisms 210 (e.g., LEDs) into the substrates, including glue, stitches, adhesive tape, snaps, hook and loop fasteners, weaving the light delivery mechanisms into the fabric itself, or printing the light delivery mechanisms 210 onto a flexible substrate embedded in or mounted on the fabric.

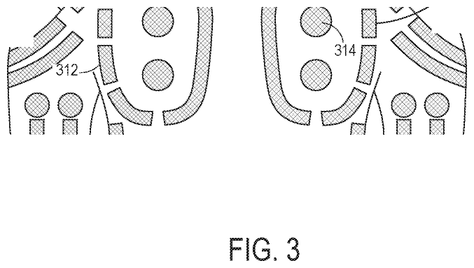

FIG. 3 illustrates a substrate 300 incorporating a system for inhibiting microbial growth according to an example embodiment. Substrate 300 is configured to deliver light to target areas in the upper body including the back, sides or chest, e.g., positioned over the lungs. Major components of the substrate include the light source (e.g., LEDs or low-level lasers), conductors/wires 302 to connect electrical components, sensors, including light sensors to modulate dosing, stretch sensor(s) to modulate dosing based on the surface area of the targeted region. A variety of light sources are available to fit different target areas, including multi-diode/multi-chipset pucks, multi-diode/multi-chipset strips to provide concentrated light delivery to a target area, printed micro LEDs on a form-fitting, flexible substrate, flexible oLED technology embedded in fabric 312, grid-embedded LED strips, and face generated low-level laser technology grids 314.

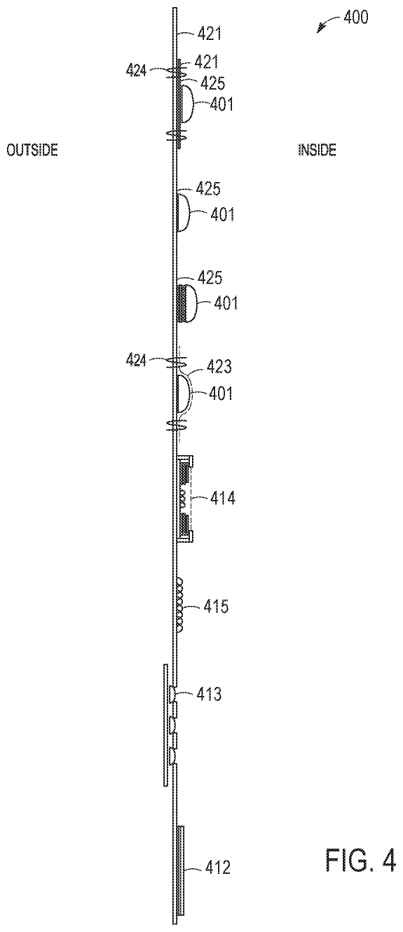

FIG. 4 is a block cross sectional representation 400 of multiple example arrangements of light sources 401 supported by a substrate 421. Major components of the substrate are meant as a balance for cost, durability, performance, comfort, design, usability, and/or waterproofing. These components include the light source 401 (e.g., LED strips or low level lasers), flexible oLED technology embedded in fabric 421, grid embedded LED strips 413, and face-generated low-level laser technology grids 414. The light source 401 incorporates a variety of technologies, including micro LEDs, oLEDs, grid-embedded LEDs, and face-generated LLLT, to target a variety of bodily regions, substrate, e.g., fabric 421, hook and loop connection 422, sheer fabric sleeve 423 for threading lights, stitching 424, e.g., thread, conductive thread, conductive ink, conductive epoxy, conductive solder, flexible thread, adhesive 425, e.g., tape, glue, heat, fusion, deposition, or other means for conducting electricity to the light sources.

The following list of components may be incorporated into one or more substrates. Some of the components are shown in FIG. 4 with reference numbers. Additional components include sensors described above associated with localized PBM to enhance NO and/or iNOS production may also he incorporated into substrates or separately supported about a human body at operable positions.
401—light source in the red/near IR wavelength
conductor/wiring/thread-/elastic, conductive ink, conductive epoxy, conductive solder.
light sensor/other sensors
quick connect
microcontroller
battery or super capacitor
energy harvester
stretch sensor
multidiode/multichipset puck
multidiode/multichipset strip
printed flexible microLED—red/near it wavelength
412—oLED fabric—red/near ir wavelength—same as claim one
413—grid embedded LED—red/near ir wavelength—same as claim one
414—Face generated LLLT—red/near ir wavelength—same as claim one
415—microLED, OLED, grid embedded, Face gen LLLT red/near ir wavelength—same as claim one [0063]
421—substrate
424—stitching
425—adhesive

Representation 400 illustrates various groupings from top to bottom of components on substrate 421. A first grouping includes stitching 424, adhesive 425, and light source 401. A next grouping just includes light source 401 supported on substrate 421 by adhesive 425. Note that each substrate may have multiple of one type of grouping in an array, or a mixture of groupings.

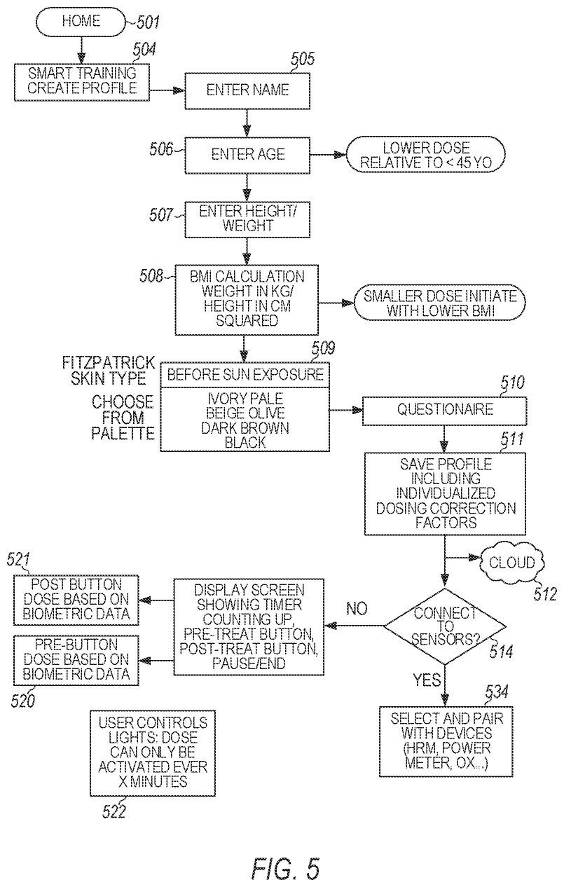

FIG. 5 is a flowchart illustrating a computer implemented method 500 use and operation of a substrate for light having an antimicrobial effect according to an example embodiment.

Enter a profile or if there is no profile, a create profile operation 504 is executed to enter a name at 505. enter an age at 506, enter a height and weight at 507, optionally BMI 508, and a skin type at 509. The skin type may be a Fitzpatrick skin type and a palate of ivory pale, beige olive, and dark Brown/Black may be selected, and based on type before sun exposure. Lower doses of light may be provided for light recipients less than a certain age, such as 45 years for humans. Smaller doses may be used with lower body mass index (BMI), but may be modified. Doses may be increased with darker skin types. A questionnaire may be provided at 510. The profile is saved at 511 and may be stored in cloud based resources 512.

The substrate is then ready 513. A first check at operation 514 is made to determine whether any sensors are associated with the substrate. A display screen may be provided at 519 to show a timer counting up, a pre treat button, a post treat button and a pause/end button. Selection of pre treat is illustrated at 520. Selection of post treat is illustrated at 521. At operation 522 the light dose may be based on a perception of virtual power. The virtual power can be estimated based on the information including parameters such as weight, height, etc. There may be a maximum dose of X joules and activation may occur every X minutes. The dose may be tailored to the stored profile.

At operation 514, if sensors are associated with the substrate pairing of the sensors with the controller may be performed at operation 534. Typical sensors include HR monitor, power meter, oxygen sensor, and others. In one embodiment, doses may be provided in response to avg power >75% for 10 mins-Power<75% but >50% of FTP for >20 mins. Similarly, data from an HR monitor and or PM and O2 sensor may be used as indicated at operation 544. Treatment initiated based on biometric data, e.g.) −75% of max HR for 10 mins-HR<75% but >50% for 20 mins.

In one embodiment a machine learning classifier may be trained based on the following metrics, which will be captured by sensors, to perform early detection of COVID-19 infection. The sensors so utilized may include one or more of near-infrared spectroscopy sensors to measure nitric oxide, blood oxygenation, muscle oxygenation sensors, heart rate and heart rate variability sensors, temperature sensors to measure body temperature, audio sensors to measure auscultation of the lungs for findings of microbial effects, such as COVID-19 effects, as well as detecting respiratory rate.

In one embodiment, sensor data may be collected from patients confirmed as infected with a selected microbe, such as COVID-19. The collected data will be labeled, such as by one or more persons, and used to train a machine learning classifier. Multiple different classifier models may be trained based on different combination of one data from one or more such sensors. Model accuracy may be improved by training with additional data as it is collected.

Patients may also receive photobiomodulation therapy to assess their response to therapy.

High risk personnel, such as ER physicians may also be used to collect and analyze the resulting sensor data to predict microbial effects before traditional screening is able to detect. Personnel that later contract a microbe may be identified and their data may labelled and used as training data. Such training data may be temporally filtered based on sensor data, specific times or time ranges prior to positive test results, or otherwise to train multiple different models to predict infection with various levels of confidence.

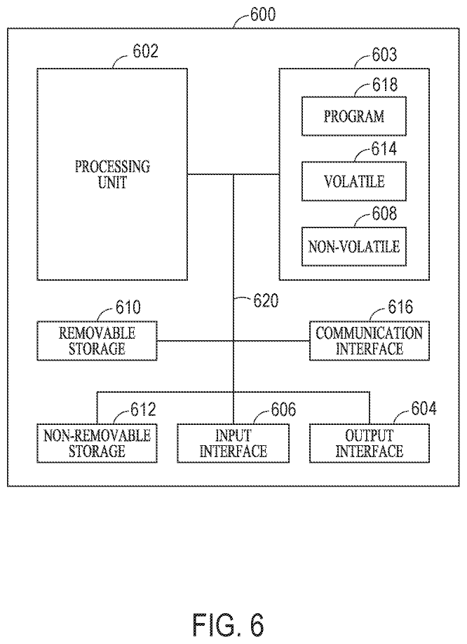

FIG. 6 is a block diagram illustrating circuitry for controlling lights to enhance performance of selected muscle groups and performing methods according to example embodiments. All components need not be used in various embodiments.

One example computing device in the form of a computer 600 may include a processing unit 602, memory 603, removable storage 610, and non-removable storage 612. Although the example computing device is illustrated and described as computer 600, the computing device may be in different forms in different embodiments. For example, the computing device may instead be a smartphone, a tablet, smartwatch, or other computing device including the same or similar elements as illustrated and described with regard to FIG. 6. Devices, such as smartphones, tablets, and smartwatches, are generally collectively referred to as mobile devices or user equipment. Such devices may be worn separately from, or integrated into the substrate incorporating light delivering devices. Further, although the various data storage elements are illustrated as part of the computer 600, the storage may also or alternatively include cloud-based storage accessible via a network, such as the Internet or server based storage.

Memory 603 may include volatile memory 614 and non-volatile memory 609. Computer 600 may include or have access to a computing environment that includes—a variety of computer-readable media, such as volatile memory 614 and non-volatile memory 608, removable storage 610 and non-removable storage 612. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 600 may include or have access to a computing environment that includes input interface 606, output interface 604, and a communication interface 616. Output interface 604 may include a display device, such as a touchscreen, that also may serve as an input device. The input interface 606 may include one or more of a touchscreen, touchpad, mouse, keyboard, camera, one or more device-specific buttons, one or more sensors integrated within or coupled via wired or wireless data connections to the computer 600, and other input devices. The computer may operate in a networked environment using a communication connection to connect to one or more remote computers, such as database servers. The remote computer may include a personal computer (PC), server, router, network PC, a peer device or other common DFD network switch, or the like. The communication connection may include a Local Area Network (LAN), a Wide Area Network (WAN), cellular, WiFi, Bluetooth, or other networks. According to one embodiment, the various components of computer 600 are connected with a system bus 620.

Computer-readable instructions stored on a computer-readable medium are executable by the processing unit 602 of the computer 600, such as a program 618. The program 618 in some embodiments comprises software that, when executed by the processing unit 602, performs operations according to any of the embodiments included herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 618 may be used to cause processing unit 602 to perform one or more methods or algorithms described herein.

The use of the substrate allows for a dose of red and/or infrared light that increases NO and/or iNOS, and/or decreases IL-6 systemically or locally, in a mammal such as a human patient, having or suspected of having a microbial infection. For example, for microbes that infect the lung, one or more substrates, e.g., substrates with a width and length 6×6 inches, are applied to the back, side and/or ribs of a patient so as to deliver an anti-microbial amount of red and/or infrared light. The light energy may not necessarily directly reach the lungs (site of infection) but will result in an increase in NO and/or iNOS, e.g., in any endothelial cells or muscle tissues that are exposed. In one embodiment, the light energy may be delivered in one or more 10 minutes intervals up to about 35 to 45 minute intervals. The time between intervals can vary from about 10 minutes to 35 to 45 minutes. The substrate may include sensors to monitor physiologic conditions, e.g., measure nitric oxide, blood oxygenation, muscle oxygenation, heart rate and/or heart rate variability, measure body temperature with a temperature sensor, and/or measure auscultation of the lungs, e.g., using an audio or accelerometer, for instance, for findings of COVID-19 as well as detecting respiratory rate. Also provided is the use of machine learning-based classification to predict, e.g., indirectly, infection such as viral infection, or an increase in viral replication, based on sensor data.

### Exemplary Embodiments

In one embodiment, the disclosure provides for a method comprising: applying one or more substrates containing one or more light emitting devices to skin of a mammal in need of antimicrobial therapy; and controlling the light emitting devices to deliver an anti-microbial amount of light to the mammal, wherein the light has a wavelength in the range of 400 nm to 1300 nm and a total dose of irradiance that is 2 mW/cm2 to 100m W/cm2. In one embodiment, the total light dose is limited to that which continues to provide an antimicrobial effect as determined by a biphasic dose response curve and thermal relaxation time. In one embodiment, the light dose is controlled based on the mammal's characteristics comprising one or more of skin color, body type, body mass index, or reflectance. In one embodiment, the one or more light emitting devices are attached to or embedded into the one or more substrates in close proximity to the mammal's body part comprising target tissue or body area. In one embodiment, the one or more light emitting devices are held in close proximity to the skin of the mammal with one or more of stitching, hook and loop fasteners, magnetic strips, adhesive, heat activated seam tape, compression, and embedding within the substrate. In one embodiment, the microbe is a bacterium. In one embodiment, the microbe is a virus. In one embodiment, the mammal is a human. In one embodiment, green light is directed at the eyes to relieve symptoms of migraine or headache. In one embodiment, one or more substrates have one or more light emitting devices. In one embodiment, phase change material is placed between the light source and the user's skin and light is used to trigger vasodilation and rapid reduction of body temperature.

Further provided is a machine-readable storage device having instructions for execution by a processor of a machine to cause the processor to perform operations to perform a method of applying an amount of antimicrobial Behr via a substrate supporting light emitting devices positioned to direct light to a selected body part of a mammal, the operations comprising: controlling the light emitting devices to deliver an anti-microbial amount of light to the body part; and wherein the light is controlled to have a wavelength in the range of 400 nm to 1300 nm and a total dose of irradiance from 2 mW-100 MW/cm2. In one embodiment, the total light dose is limited to that which continues to provide an antimicrobial effect as determined by a biphasic dose response curve and thermal relaxation time.

Also provided is a method to prevent, inhibit or treat microbial infection in a mammal, comprising: applying one or more substrates containing one or more light emitting devices to skin of a mammal having a microbial infection or exposed to a source having the microbe; and controlling the light emitting devices to deliver an anti-microbial amount of light to the mammal, wherein the light has a wavelength in the range of 400 nm to 1300 nm and a total dose of irradiance that is no more than 100 mW/cm2. In one embodiment, the method includes monitoring one or more parameters in the mammal exposed to the source having the microbe to determine if or when light delivery is initiated. In one embodiment, the method includes monitoring one or more parameters in the mammal having the microbial infection to determine if or when light delivery is initiated. In one embodiment, one parameter is an increase in body temperature. In one embodiment, the method further includes monitoring one or more parameters in the mammal having the microbial infection or exposed to the source having the microbe to determine changes in the one or more parameters after light delivery. In one embodiment, green light is directed at the eyes to relieve symptoms of migraine or headache. In one embodiment, one or more substrates have one or more light emitting devices. In one embodiment, phase change material is placed between the light source and the user's skin and light is used to trigger vasodilation and rapid reduction of body temperature.

The disclosure provides a method comprising: subjecting a mammalian physiological fluid sample ex vivo to an antimicrobial amount of light emitted from one or more light emitting devices, wherein the light has a wavelength in the range of 400 nm to 1300 nm.

### Example 1

1. A method comprising: applying a substrate containing light emitting devices to a body part of a human; and controlling the light emitting devices to deliver light to the body part, wherein the light has a wavelength in the range of 600 nm to 1300 nm and is delivered in an antimicrobial amount.

2. A method comprising: applying a substrate containing light emitting devices to a body part of a human; and controlling the light emitting devices to deliver light to the body part, wherein the light has a wavelength in the range of 400 nm to 1300 nm and is delivered in an antimicrobial amount.

3. A substrate and method that delivers light in the red and/or near-infrared spectrum in the range of 600 nm-700 or 700-1300 nm, which may be used concurrently or at different time periods and the Red/IR light may be controlled independently.

4. The method of any of the previous examples wherein the red light is delivered at an irradiance from 2 mW-600 mW/cm2

5. The method of any of the previous examples wherein the red light is delivered at an irradiance of at least 5 mW/cm2

6. The method of any of the previous examples wherein the red light is delivered at an irradiance of at least 10 mW/cm2

7. The method of any of the previous examples wherein the near infrared light is delivered at an irradiance from 2 mW-600 mW/cm2.

8. The method of any of the previous examples wherein the near infrared light is delivered at an irradiance of at least 5 mW/cm2.

9. The method of any of the previous examples wherein the near infrared light is delivered at an irradiance of at least 10 mW/cm2.

10. The method of any of the previous examples wherein the total light dose is limited to only that which provides an antimicrobial effect as determined by the biphasic dose response curve and thermal relaxation time. One example of a biphasic dose response includes: A light dose of less than X joules does not trigger an antimicrobial effect; light dose between X and Y joules triggers an antimicrobial effect; light dose above Y joules is detrimental to the human. X and Y may be determined empirically.

11. The method of any of the previous examples wherein the light dose is controlled in accordance with a protocol that is used to capture an individual's characteristics such as skin color, body type, body mass index, and; or reflectance. Skin color, skin thickness, subcutaneous fat affect absorption and reflection of light. Therefore, the total dose delivered is adjusted to account for the effect of these parameters on light absorption and reflection.

12. The method in example 11, wherein an individual's dose is higher if the individual's skin color is darker, e.g. darker skin may require higher irradiance than a lighter skin individual depending on the given light wavelength. A certain amount of energy is referred to as a dose. The length of time required to deliver a certain amount of energy depends on the power output of the lights (irradiance).

13. The method of any of the previous examples wherein the device is attached to the back of a patient.

14. The method of any of the previous examples wherein the light source is in a substrate and is held in the substrate with one or more of stitching, hook and loop fasteners, magnetic strips, adhesive, heat activated seam tape, compression, and embedded within the textile itself

15. The method of any of the previous examples wherein the light source is held at a near constant distance between the light source and the skin and aided by a thin fabric or film between the skin and light source

16. The method of any of the previous examples wherein an individual may be provided a higher dose per area based on disease symptoms

17. The method of any of the previous examples, wherein the controller is be set to treat at pre-specified parameters for prolonged use.

18. The method of any of the previous examples, where an onboard microcontroller is in sync with an external device s light therapy would be delivered when a certain physiologic measure is detected or a threshold is reached.

19. The method of any of the previous examples, wherein a power supply, such as a battery, super-capacitor, or energy harvester is integrated into or affixed to the substrate, be chargeable, and also removable.

20. The method of any of the previous examples wherein the light source is low level light therapy, which is selectively pulsed, super-pulsed, continuous, or applied sequentially to target tissue as determined by physical state of the individual

21. The method of any of the previous examples, wherein the individual has an active microbial infection.

22. The method of any of the previous examples, wherein the light source is in close proximity and targets related chromophores in but not limited to the epidermis and associated appendages, reticular and papillary dermis, blood vessels in the dermis and other subcutaneous tissues, fascial plane and associated tissue, hematologic cells, as well as skeletal muscle and associated cells and blood vessels.

23. The method of any of the previous examples, wherein the goal of target tissue being treated with various wavelengths induces an increase in creatinine kinase, lactate and lactic acid, and other breakdown byproducts, nitrite-nitric oxide modulation, cytochrome C oxidase or other mitochondrial proteins, delayed gene transcription by way of oxidative stress, as well as other targets in the tissue.

24. The method of any of the previous examples, wherein the therapy parameters are adjusted or preset prior to an activity, or adjustable via a separate microprocessor or controller and limited/controlled by a maximum amount of energy delivered in a given time period and time interval between light applications.

25. The method in example 24, wherein the light parameters are adjusted and monitored by devices such as an external application on a smart device, computer, and/or interface with, for example, a heart rate monitor, power meter or a proprietary device.

29. The method of any of the previous examples, wherein the lights are encased into a reflective shield to allow unidirectional light application to the skin and prevent unnecessary treatment to other body parts including the eye.

26. The method of any of the previous examples, wherein the method may also include application or administration of topical lotion, cream, gel, ointment, or fluid that could include topical Nitric oxide stimulators, reactive oxygen scavengers, creatine, or electron transport chain enhancers.

Nitric oxide stimulators could include nitric oxide precursors in the nitric oxide stimulator pathway.

Reactive oxygen scavengers could include vitamins with antioxidant activity, tetrahydrocurcumin, curcumin.

Creatine to allow adequate storage and precursors for the ATP system.

Niacinamide for adequate storage/modulation of the precursor molecule NADH.

27. The method of any of the previous examples, wherein sensors are utilized to monitor cutaneous temperature to provide automated discontinuation if pre-specified temperatures are reached.

32. The method of any of the previous examples, wherein the calculated safe and optimal dose is measured by integrated stretch sensors, which may be used to calculate the surface area of the treated area is determined.

28. The method of any of the previous examples wherein the parameters of light delivery can be adjusted by a processing unit

29. The method of any of the previous examples wherein the amount of energy delivered is determined by a processing unit to account for number of light sources, surface area, battery level, and/or user variability

30. The method of any of the previous examples, wherein a local network is created between multiple substrates to coordinate light application.

31. The method of example 30 wherein the local network comprises a mesh network.

32. The method of any of the previous examples, wherein the substrate is a flexible, compressible, stretchable substrate adjoined by stretchable wiring to prevent stress on wires and connections, which may include stretchable stitches and/or stress relief features between circuitry and wiring and/or seam tape for water resistance and protection of circuitry

33. The method of any of the previous examples, wherein a panel design of lights is affixed to the substrate separately. The panel design includes heat activated seam tape to compression flexible fabric that is stitched to the substrate separately to allow flexibility of the panel and relieve mechanical stress on the light device.

34. The individual dosing parameters of the collective data from activity can be logged and uploaded to a database for future use.

### Example 2

1. A method comprising: applying a substrate containing light emitting devices to the body of a mammal; and controlling the light emitting devices to deliver light to the body such that the light has an antimicrobial effect on the mammal, wherein the light has a wavelength in the range of 620 nm to 1300 nm.

2. A method comprising: applying a substrate containing light emitting devices to a body part of a mammal; and controlling the light emitting devices to deliver light to the body part such that the light has an antimicrobial effect on the mammal, wherein the light has a wavelength in the range of 400 nm to 1300 nm.

3. An antimicrobial wearable device and method that delivers light in the red and/or near-infrared spectrum in the range of 620 nm-1300 nm.

4. The method of any of the previous examples wherein the red light is delivered at an irradiance from 0.5 mW-300 mW/cm2

5. The method of any of the previous examples wherein the red light is delivered at an irradiance of at least 1 mW/cm2

6. The method of any of the previous examples wherein the red light is delivered at an irradiance of at least 2 mW/cm2

7. The method of any of the previous examples wherein the near infrared light is delivered at an irradiance from 1 mW/cm2-300 mW/cm2

8. The method of any of the previous examples wherein the near infrared light is delivered at an irradiance of at least 3 mW/cm2

9. The method of any of the previous examples wherein the near infrared light is delivered at an irradiance of at least 5 mW/cm2.

9A. The method of any of the previous examples wherein the near infrared light is delivered in pulses at a frequency between 10 Hz and 10,000 Hz.

9B. The method of any of the previous examples wherein the red light is delivered in pulses at a frequency between 10 Hz and 10,000 Hz, e.g., 50 to 3,000 Hz or 50 to 500 Hz.

10. The method of any of the previous examples wherein the total light dose would not exceed current scientific understanding dosing per body system. For example, current understanding is that in excess of 100 mW/cm2, e.g., per treatment, is probably detrimental based on current scientific literature.

11. The method of any of the previous examples wherein the light dose would be more specifically determined by an individual's characteristics such as skin color, reflectance, subcutaneous fat, and/or body size.

12. The method in example 9, wherein an individual's dose would be higher if their skin color is darker, e.g., darker skin may require higher irradiance than a lighter skin individual.

13. The method of any of the previous examples wherein the device would be attached to, or embedded into a garment or wearable device in close proximity to an individual's targeted tissue or body area.

14. The method of any of the previous examples wherein the light source is held in close proximity with stitching, velcro, magnets, adhesive, compression, or could also be embedded within the wearable device. The use of phase change material between the lights and the skin to regulate the temperature of the device as well as the core temperature of the subject can be added, and this regulation of temperature can be beneficial to reduce fever and fatigue and improve athletic performance.

15. The method of any of the previous examples wherein the light source would be held at a near constant distance between the light source and the skin and aided by a thin fabric or film between the body and the light.

16. The method of any of the previous claims where a microcontroller is used to adapt light delivery to several factors, including individual skin color, reflectance, body composition, target tissue.

17, The method of any of the previous examples wherein light delivery is accompanied by nitrate supplements to fuel nitric oxide generation.

18. The method of any of the previous examples wherein the controller may be set to treat at prespecified parameters for prolonged use.

19. The method of any of the previous examples, wherein an onboard microcontroller is in sync with onboard or external sensors to determine the appropriate light delivery parameters based on feedback, such as near infrared spectroscopy device, muscle oxygenation, pulse oximetry, or other physiologic markers.

20. The method of any of the previous examples, wherein a power supply, such as a battery, super-capacitor, energy harvester, or external power supply would be integrated into the wearable device, be chargeable, and also removable to allow cleansing of the device.

21. The method of any of the previous examples wherein the light source is low level light therapy, which could be pulsed, super-pulsed, continuous, or applied sequentially to target tissue as determined by treatment goals, targeted tissues, and physical state of the mammalian recipient.

23. The method of any of the previous examples, wherein the light source is in close proximity and targets related structure in but not limited to the epidermis and associated appendages, reticular and papillary dermis, blood vessels in the dermis and other subcutaneous tissues, fascial plane and associated tissue, hematologic cells, cardiopulmonary system, as well as skeletal muscle and associated cells and blood vessels.

24. The method of any of the previous examples, wherein the light delivery induces a measurable physiologic benefit including but not limited to biochemical markers such as CK, lactate and lactic acid, and other breakdown byproducts, nitrite-nitric oxide stimulation, cytochrome C oxidase, superoxide dismutase, catalase and other mitochondrial proteins, as well as other targets in the tissue.

25. The method of any of the previous examples, wherein the light delivery results in:
decreased duration of viral infection and/or decreased need for medical intervention and/or decreased time to recover from illness and/or increased tissue oxygenation and/or increased generation of nitric oxide and/or inhibition of viral replication and/or treatment of High Altitude Pulmonary Edema (HAPE) and/or decreased development of neutrophilic lung inflammation, leukocyte count, mast cell degradation and/or MPO activities and/or decreased microvascular lung permeability in the parenchyma and intrapulmonary bronchi and/or decreased levels of inflammatory cytokines and/or increased anti-inflammatory cytokines in the lungs and/or decreased systemic inflammation and/or decreased myocardial dysfunction.

26. The method of any of the previous examples, wherein the total irradiance dose would not exceed current standards known to be deleterious or harmful to the exposed tissue based on current research as depicted by the biphasic response as determined by thermal relaxation time.

27. The method of any of the previous examples, wherein the light therapy would be applied periodically at a prespecified interval at prespecified dosage parameters.

28. The method of any of the previous examples, wherein the wearable could include but not be limited to: compression wrap, elastic band, shirt, gown, any other iteration with wearable light source for antimicrobial therapy.

29. The method of any of the previous examples, wherein the therapy parameters may be adjusted or preset prior to treatment, or may be adjustable via a separate microprocessor or controller.

30. The method in claim 27, wherein the technology may be adjusted by devices such as external application on a smart device, computer, controller, and/or interface with a near infrared spectroscopy device, muscle oximeter, pulse oximeter or a proprietary device.

31. The method of any of the previous examples, wherein light therapy technology could be embedded into compression wrap to be utilized for both recovery and anti-microbial therapy of select tissue groups.

32. The method of any of the previous examples, wherein the lights would be encased into a reflective shield to allow unidirectional light application to the skin and prevent unnecessary treatment to other body parts including the eye.

33. The method of any of the previous examples, wherein the therapy device may be enhanced by:
Application of topical lotion, cream gel, ointment, or fluid
The application of Nitric oxide stimulators, reactive oxygen scavengers, creatine, or electron transport chain enhancers
Nitric oxide stimulators would include nitric oxide precursors in the nitric oxide stimulator pathway
Reactive oxygen scavengers could include vitamins with antioxidant activity, tetrahydrocurcumin, curcumin.
Creatine to allow adequate storage and precursors for the ATP system
Niacinamide for adequate storage of and precursor molecule NADH.

34. The method of any of the previous examples, wherein a temperature sensor could be utilized to allow monitoring of cutaneous temperature to allow automated treatment discontinuation if pre-specified. temperatures are reached.

35. The method of any of the previous examples, wherein the calculated safe and optimal dose is measured by integrated stretch sensors, which may be used to calculate the surface area of the treated area is determined.

36. An anti-microbial therapy wearable device comprising: a substrate adapted to be worn proximate a body system for which therapy is desired; an array of light producing elements positioned to direct light to the body, and held in place by the substrate; and a controller to control the array of light producing elements to provide therapeutic light to the body, wherein the light has a wavelength in the range of 400 nm to 1300 nm.

37. The device of example 36 wherein the substrate comprises clothing.

38. The device of example 37 wherein the array of light producing elements is held in place by the use of stitching, velcro, magnets, adhesive, compression, or is embedded within the clothing itself.

39. The device of example 36 and further comprising at least one near infrared spectroscopy device such as an external muscle oximeter or pulse oximeter coupled to the controller.

40. The device of example 36 wherein the controller controls the light source to be pulsed, super-pulsed, continuous, or applied sequentially to target tissue as determined by therapeutic goals (recovery or antiviral), and physical state of the participant.

41. The device of example 36 wherein the controller controls less than all of the light producing element to provide light corresponding to a shape of the targeted body system.

42. The device of example 36 Wherein the substrate comprises compression wrap, shirt, gown, sleeve, or elastic band.

43. A method comprising: applying a substrate containing light emitting devices to the body of a mammal; and controlling the light emitting devices to deliver light to the body such that the light relieves symptoms of a migraine or a headache.

44. The method of example 43 wherein the light is green light, e.g., from at 520 nm to about 560 nm.

45. The method of any the previous examples wherein phase change material is placed between the height source and the user's skin and light is used to trigger vasodilation and rapid reduction of body temperature.

Although a few embodiments have been described in detail above, other modifications are possible. For example, the logic flows depicted in the figures do not require the particular order shown, or sequential order, to achieve desirable results. Other steps may he provided, or steps may be eliminated, from the described flows, and other components may be added to, or removed from, the described systems. Other embodiments may be within the scope of the following claims.

What is claimed is:

1. A method comprising:
applying one or more substrates containing one or more light emitting devices to skin of a mammal in need of antimicrobial therapy; and
controlling the light emitting devices to deliver an anti-microbial. amount of light to the mammal, wherein the light has a wavelength in the range of 400 nm to 1300 nm and a total dose of irradiance that is up to 100 mW/cm2 or from 2 mW/cm2 to 100 mW/cm2.

2. The method of claim 1 wherein the light has a wavelength in the range of 600 nm to 1300 nm.

3. The method of claim 1 wherein the red light is delivered at an irradiance from 2 mW-100 mW/cm2 or is delivered at an irradiance of at least 5 mW/cm2.

4. The method of claim 1 wherein the total light dose is limited to that which continues to provide an antimicrobial effect as determined by a biphasic dose response curve and thermal relaxation time.

5. The method of claim 1 wherein the light dose is controlled based on the mammal's characteristics comprising one or more of skin color, body type, body mass index, or reflectance.

6. The method of claim 5 wherein the dose is higher if the skin color of the mammal is darker, wherein the dose is determined as a function of irradiance power, wavelength, and length of time.

7. The method of claim 1 wherein the one or more light emitting devices are attached to or embedded into the one or more substrates in close proximity to the mammal's body part comprising target tissue or body area.

8. The method of claim 7 wherein the one or more light emitting devices are held in close proximity to the skin of the mammal with one or more of stitching, hook and loop fasteners, magnetic strips, adhesive, heat activated seam tape, compression, and embedding within the substrate.

9. The method of claim 1 wherein the microbe is a bacterium or virus.

10. The method of claim 1 wherein the mammal is a human.

11. The method of claim 1 wherein the one or more substrates are applied to the back or sides of the chest of the mammal.

12. A machine-readable storage device having instructions for execution by a processor of a machine to cause the processor to perform operations to perform a method of applying an amount of antimicrobial light via a substrate supporting light emitting devices positioned to direct light to a selected body part of a mammal, the operations comprising:
controlling the light emitting devices to deliver an anti-microbial amount of light to the body part; and
wherein the light is controlled to have a wavelength in the range of 400 nm to 1300 nm and a total dose of irradiance of from 2 mW-100 mW/cm2.

13. The machine-readable storage device of claim 12 wherein the total light dose is limited to that which continues to provide an antimicrobial effect as determined by a biphasic dose response curve and thermal relaxation time.

14. A method to prevent, inhibit or treat microbial infection in a mammal, comprising:
applying one or more substrates containing one or more light emitting devices to skin of a mammal having a microbial infection or exposed to a source having the microbe; and
controlling the light emitting devices to deliver an anti-microbial amount of light to the mammal, wherein the light has a wavelength in the range of 400 nm to 1300 nm and a total dose of irradiance that is no more than 100 mW/cm2.

15. The method of claim 14 wherein the source is a different mammal infected with the microbe.

16. The method of claim 14 wherein the mammal having the microbial infection or exposed to the source having the microbe is a human.

17. The method of claim 14 further comprising monitoring one or more parameters in the mammal exposed to the source having the microbe to determine if or when light delivery is initiated, wherein optionally one parameter is an increase in both temperature.

18. The method of claim 14 further comprising monitoring one or more parameters in the mammal having the microbial infection to determine if or when light delivery is initiated, wherein optionally one parameter is an increase in body temperature.

19. The method of claim 14 further comprising monitoring one or more parameters in the mammal having the microbial infection or exposed to the source having the microbe to determine changes in the one or more parameters after light delivery.

20. The method of claim 1 wherein green light is directed at the eyes to relieve symptoms of migraine or headache.
